(19) United States
(12) Patent Application Publication
(10) Pub. No.: US 2011/0097293 A1
(43) Pub. Date: Apr. 28, 2011

(54) MULTIPLE COMPONENT SYSTEMS AND METHODS FOR HAIR RELAXATION OF VARIABLE STRENGTH

(76) Inventors: Gary Grey, New York, NY (US); Mark Szelast, Walnutport, PA (US)

(21) Appl. No.: 12/606,810

(22) Filed: Oct. 27, 2009

(51) Int. Cl. A61K 8/19; A61Q 5/04

(52) U.S. Cl. 424/70.2

(57) ABSTRACT

Systems and related methods for straightening or relaxing human hair. The components of the systems and methods include a formulated relaxer cream or relaxer precursor cream that contains a level of alkali metal hydroxide or alkali earth metal hydroxide and an additional diluent cream of similar viscosity and consistency, yet lacking the alkali ingredient, that serves to dilute the strength of the hair-straightening admixture without any change in viscosity that may render the mixture useless or even harmful. A no-lye system will also include an activator solution, such as guanidine carbonate, to react with the alkali earth metal hydroxide. The components of the systems are mixed prior to application to the user's hair. The systems and methods provide a gentler and safer approach to straightening hair for users whose hair possesses low levels of curl or finer hair texture, for users seeking only a lesser degree of hair straightening.

### TECHNICAL FIELD

The invention relates generally to compositions, systems, kits and methods for straightening hair, particularly human hair. More particularly, the invention is directed to gentle and effective methods, compositions, systems and kits for variable strength lye and no-lye hair relaxers that provide a consumer with an ability to decrease the strength of the relaxer to a suitably low effective level at the time of application. These systems and methods permit consumers with a low degree of curl/kink to their hair, consumers with hair of fine texture, and consumers who desire a lesser degree of straightness to their hair to adjust their hair relaxer to a decreased strength to avoid excessive straightening, potential deleterious effects or damage to the hair that may occur with commercially available hair relaxers of fixed and/or greater strengths. The present systems, methods and compositions also allow the user to safely reduce the concentration of the relaxing agents in their hair relaxer in a controlled and convenient manner without impacting the consistency or effectiveness of the hair relaxers or processes.

### BACKGROUND OF THE INVENTION

Hair relaxers are used for permanent straightening of curly and/or kinky hair. The relaxers work by breaking the disulfide bonds within the hair, mechanically straightening the hair, and then reforming the disulfide bonds when the hair is in a straightened position through neutralization of the active ingredient in the relaxer, which is often a strong alkali.

There are two common types of alkali hair relaxers. The first type is known as a lye relaxer. The most commercially prevalent example contains sodium hydroxide. Lye relaxers are in cream form and contain the alkali. They are applied directly to the hair. Because of the inherent dangers of sodium hydroxide, lye relaxers are usually relegated to the professional sector of the hair care market. The second type of alkali relaxer is known as a no-lye relaxer. They make up the bulk of the home use market sector. A no-lye relaxer comprises a precursor cream which contains an alkali earth metal hydroxide, such as calcium hydroxide, which is mixed with a liquid activator that contains a strong nitrogenous base, such as guanidine carbonate. The activator liquid and the precursor cream are mixed just prior to use, and the chemical reaction between these components, for example, the calcium hydroxide in the precursor cream and guanidine carbonate in the liquid, produces the hair relaxing agent in activated form, in this case, guanidine hydroxide, which becomes the active alkali in treating the hair.

No-lye relaxers are generally considered to be safer for home use, but they still present potential dangers if not used properly. Over processing by leaving the activated cream on the hair too long can cause permanent damage to the hair, and contact of the relaxer with the scalp or other skin areas can cause irritation or burns. It is generally accepted that the safest and most effective way to straighten human hair is to use the mildest type and strength of relaxer for a given user's hair type. The degree of curl/kink in the hair, the desired level of straightness of the hair, and the coarse or fine nature of the hair's texture should dictate the period of time the relaxer remains on the hair before it is neutralized and the necessary strength of the relaxer. Manufacturers of relaxers may make different strengths of a lye relaxer and will often vary the amount of the activator solution in the relaxer kit to vary the strength of no-lye relaxer systems. However, even the mildest strength of relaxer in either system may still be stronger than is needed for straightening by users with particularly fine hair, a low amount of curl or kink, or who are seeking a lesser degree of straightness to the hair.

### SUMMARY OF THE INVENTION

The present invention is, therefore, directed to systems, kits and methods for effecting gentle and effective straightening of curly or kinky hair by use of a variable strength lye or no-lye alkali-containing relaxer to enable the user having or wanting a low degree of curl/kink or having fine textured hair to decrease the strength of the relaxer to a lower effective level than the relaxer would originally have.

In a no-lye system, the alkali-containing precursor cream is mixed with an activator and that mixture produces an active hair relaxing agent (for example, guanidine hydroxide). A lye system does not require a separate activator to produce the active relaxing agent, which is, for example, sodium hydroxide or lithium hydroxide.

The strength of either the lye or no-lye relaxer would be varied by providing an extra amount of a diluent cream, generally having the same ingredients in the same concentrations as the lye or no-lye alkali-containing relaxer cream or relaxing precursor cream, respectively, so as to achieve the same product viscosity, but which contains no alkali metal hydroxide (for example, sodium hydroxide) or alkali earth metal hydroxide (for example, no calcium hydroxide). This diluent cream may be included as a component in the kit to be mixed directly with the lye relaxer in the lye relaxer system to dilute the level of active alkali (such as sodium hydroxide). In the no-lye system, the diluent cream may be included as a component in the kit to be mixed with the relaxing precursor cream/activator mix after the guanidine hydroxide has been formed in situ, also as to dilute the level of the active alkali and make the relaxer safer and more easily controlled.

The invention concerns systems and related methods for straightening or relaxing human hair. The components of the lye relaxer system and method include a formulated relaxer cream that contains a level of alkali metal hydroxide and an additional diluent cream of similar viscosity and consistency, yet lacks the active metal hydroxide ingredient, serving to dilute the strength of the hair-straightening mixture without changing the viscosity, as change in viscosity may render the mixture useless or even harmful. The components of the no-lye relaxer system and method include an activator solution with a strong nitrogenous base, along with a formulated relaxer precursor cream that contains a level of alkali earth metal hydroxide and an additional diluent cream of similar viscosity and consistency, only lacking the active alkali earth metal hydroxide ingredient, that serves to dilute the strength of the hair-straightening admixture without changes in viscosity.

The components of the systems may be mixed just prior to the application to the user's hair. The systems and methods enable a gentler and safer approach to straightening hair for users whose hair possesses low levels of curl, users who have particularly fine textured hair, users who desire only a lesser level of straightness to the hair, and for users who seek to avoid the use of harsher hair-straightening methods and formulations.

### DETAILED DESCRIPTION OF THE INVENTION

The present invention involves multi-component hair straightening systems and methods and involves varying the strength of the active hair relaxing agent. In a lye relaxer system, the first system component is a relaxer cream that preferably contains an effective concentration of alkali metal hydroxide, preferably sodium hydroxide, preferably in concentrations ranging from 0.1 to about 20% by weight, or more. The relaxer cream has ingredients in such quantities selected so as to provide the cream with a level of viscosity that makes the cream easy to shape and apply to the hair in controllable volumes, without clumping, running or dripping.

The lye relaxer system also has a second system component, which is a diluent cream containing ingredients either identical or closely similar to the ingredients of the relaxer cream, and thus possesses a viscosity and consistency equal to or highly similar to that of the relaxer cream. The key difference and exception is in that the diluent cream omits the active lye relaxing agent itself, such as sodium hydroxide, so that the diluent cream is essentially inert. The diluent cream serves instead to dilute the concentration of the lye relaxer while still generally maintaining the consistency and viscosity of the relaxer cream upon dilution.

In a no-lye relaxer system, the first system component, referred to herein as the hair relaxing precursor cream or calcium cream, has ingredients in such quantities selected so as to provide the cream with a level of viscosity that makes the cream easy to shape and apply to the hair in controllable volumes, without clumping, running or dripping. The hair relaxing precursor cream preferably contains an effective concentration of alkali earth metal hydroxide, preferably calcium hydroxide, as the active precursor, preferably in a concentration of about 6.0% by weight, but generally at concentrations ranging from 0.1% to about 20% by weight, or more. Due to its low solubility in water, the alkali earth metal hydroxide, such as calcium hydroxide, is suspended in a thick emulsion which is preferably stable under alkaline conditions. Due to the low aqueous solubility of the alkali earth metal hydroxide, such as calcium hydroxide, the relaxing precursor cream is generally ineffective as a hair relaxer by itself.

The second system component of the no-lye relaxer system is referred to herein as the activator solution. The activator solution contains a reactive agent, preferably a strong nitrogenous base, such as guanidine carbonate, which is also essentially ineffective for straightening hair when applied alone. The reactive agent, such as guanidine carbonate, derives its efficacy as an activator upon its introduction into or mixing with an alkali earth metal hydroxide relaxing precursor, such as calcium hydroxide, and the resulting reaction produces an active, functional hair relaxing agent, such as guanidine hydroxide. In a preferred embodiment of the present invention, the reaction of the guanidine carbonate with the calcium hydroxide produces guanidine hydroxide as the active relaxing agent. When the product of the mixture, guanidine hydroxide, is applied to the subject's hair, it serves as the effective hair relaxing agent, breaking the disulfide bonds within the hair and allowing the hair to be mechanically strengthened.

The activator solution in the no-lye relaxer system often contains a dye, which makes it easier for the user to determine when the activator and the cream are thoroughly mixed. It may also contain a thickening agent, one that is effective under highly alkaline conditions, to stabilize the dissolved guanidine carbonate and prevent its re-crystallization and precipitation from the solution.

The third system component of the no-lye relaxer system is a diluent cream that contains ingredients either identical, or closely similar, to the ingredients of the hair relaxing precursor cream and thus possesses a viscosity and consistency equal to or highly similar to that of the relaxing precursor cream. The important difference and exception is that the diluent cream omits the active relaxing precursor itself (such as an alkali earth metal hydroxide, preferably calcium hydroxide), so that the diluent cream is essentially inert. It instead dilutes the concentration of the active relaxing agent, while still generally maintaining the consistency and viscosity of the relaxing precursor cream upon dilution.

In both the lye relaxer and no-relaxer systems, retaining the viscosity of the cream during the hair straightening process is beneficial for several reasons. The cream should be thick enough to keep the hair in a straightened position during the processing time, but not so thick as to make the cream unduly difficult to spread over the hair, and it should not be so thin as to drip from the hair onto the scalp which could cause irritation or burning.

In each of the lye relaxer and no-lye relaxer systems, respectively, the use of the diluent cream to reduce the concentration of the hair relaxing agent thus allows for a safer and more manageable application of the relaxer while maintaining the relaxer's effectiveness and efficacy. It also broadens the applicability of the hair straightening products and systems for use by professionals or individuals who might find the concentrations of hair relaxing agents generally offered in the industry or marketplace to be overly harsh, strong or impractical for use with a subject's hair, particularly if the subject's hair only possesses a mild degree of curl or kink, if the subject's hair possesses a fine texture, or if the subject desires only a low level of straightening for the hair. The multi-component systems disclosed herein are improvements upon the commonly available hair straightening systems, methods and products.

As indicated above, therefore, the systems and compositions according to the invention can also be used, in a modified regimen, as a mild and effective way to allow those who, for style reasons, wish to only remove some of the hair curl, and not straighten the hair completely. Furthermore, the systems are gentle and effective enough across the range of fine or coarse hair textures, hair curl/kink types, and ages. Considering the reluctance of many adults to use harsher relaxers on their own or their children's hair, providing the safe diluent cream with hair straightening products or kits may serve to broaden the target market and/or promote an effective alternate or adjunct option to previously practiced methods.

As discussed above, no-lye relaxers are generally considered to be safer for home use, but they still present potential dangers if not used properly. Excessive concentrations of relaxing agents and excessive exposure of the hair to relaxing agents in either lye relaxer systems or in no-lye relaxer systems can lead to permanently damaged hair, and contact with the scalp or other skin can cause irritation and burns. It is generally accepted that the safest and most effective way to straighten one's hair is to use the mildest type of relaxer sufficient for use with one's hair type. The fine or coarse texture of the untreated hair, the desired level of straightening sought, and/or the degree of curl/kink in the untreated hair will dictate the required minimum strength of the relaxer for efficacy. Manufacturers and sellers of commercial relaxers may offer different strengths of lye relaxer products, and will often make several differentiated strengths of a no-lye relaxer, typically by varying the amount of the activator solution in the relaxer kit. However, even the mildest concentration of relaxer readily available may still be excessive, and unnecessarily risky or unsafe, for those users with only a low amount of curl or kink in their hair or with hair of finer textures. Furthermore, in no-lye relaxer systems, there may be limitations as to the effectiveness of indirectly reducing the strength of the relaxer cream and the relaxing agent based solely on varying the amount of the activator solution.

The present invention relates to variable strength lye relaxer and no-lye relaxer systems that would provide to the user with a low degree of hair curl/kink, hair of fine texture, or a desire for a lesser degree of hair straightening, the ready ability to decrease the concentration of the relaxing agent in the relaxer to the lowest, and thus most safe, effective level. This adjustment of the lye or no-lye relaxer agent concentration is accomplished by providing an extra amount of diluent cream, which contains no alkali (for example, sodium hydroxide or calcium hydroxide), in the kit to mix directly with the relaxer cream (in a lye relaxer system) or, in a no-lye relaxer system, to mix with the relaxing precursor cream/guanidine carbonate activator mix after the active or activated relaxing agent, preferably guanidine hydroxide, has been formed in situ, thus diluting the level of the active alkali and making the relaxer safer and more easily controlled. A simple dilution of the mixture with water or another solvent might accomplish a similar result, but the resulting mixture would be too thin (with application of water) or too thick, dependent on the solvent, and would increase the risk of dripping onto the scalp, not “holding” the hair straight during processing time, or making the relaxer cream unmanageable. Providing a diluting cream of a comparable viscosity to that of the relaxer cream or relaxer precursor cream allows a safe, simple, and effective way of obtaining a relaxing agent with the minimum effective strength for a given hair type.

### Examples

The following examples are provided only for the purpose of illustrating the invention and they are, therefore, not to be construed as limiting.

An embodiment of a no-lye relaxer cream comprises the following:

TABLE ONE
(NO-LYE RELAXER)
% by weight

| Cream: | % | Activator: | % |
|---|---|---|---|
| 1. Petrolatum | 10.0 | 1. Water | 72.7 |
| 2. Mineral Oil | 10.3 | 2. Guanidine Carbonate | 27.0 |
| 3. Cetearyl Alcohol | 10.0 | 3. Xanthan Gum | 0.3 |
| (and) Ceteth-20 | | | |
| 4. PPG-5-Ceteth-20 | 1.0 | | |
| 5. PEG-75 Lanolin | 0.5 | | |
| 6. Glycerin | 3.0 | | |
| 7. Water | 59.2 | | |
| 8. Calcium Hydroxide | 6.0 | | |

TABLE TWO
(LYE RELAXER)
% by weight

| Cream: | % |
|---|---|
| 1. Petrolatum | 23.0 |
| 2. Mineral Oil | 15.0 |
| 3. Cetearyl Alcohol (and) Dicetyl Phosphate | 8.0 |
| (and) Ceteth-10 Phosphate | |
| 4. Propylene Glycol | 3.0 |
| 5. Cetyl Alcohol | 1.0 |
| 6. PPG-5-Ceteth-20 | 1.0 |
| 7. PEG-75 Lanolin | 0.7 |
| 8. Water | 45.82 |
| 9. Sodium Hydroxide | 2.17 |

The variable adjustable strength relaxer system of the present invention also provides a separate additional amount of the above listed cream formulas in a separate container of diluent cream, from which the alkali ingredient is omitted (calcium hydroxide item 8 in the cream ingredient list of Table One, and sodium hydroxide, item 9 in Table Two). In the diluent cream container, the other ingredients would be increased in the same proportions as the omitted alkali ingredients in the respective cream formulas in Tables One and Two. In this manner, the viscosities of the mixed creams is preserved at a level consistent with the viscosities of the active relaxing precursor cream and the diluent cream, such that addition of diluent cream produces an end product which is less likely to clump, congeal, splash or run, and is otherwise safer and more effective than would be use of other diluents, such as water. The diluent creams in lye relaxer systems act in an analogous fashion to the diluent creams of no-lye relaxer systems.

The present invention also relates to packages, or kits, of hair-straightening products, that feature and include, for a no-lye relaxer system: an aqueous activator solution comprising a strong nitrogenous base, preferably guanidine carbonate, in a first container, a first formulated precursor cream containing an alkali earth metal hydroxide, preferably calcium hydroxide, in a second container, and a second formulated diluent cream of essentially equal viscosity to the first formulated cream without the alkali earth metal hydroxide (such as calcium hydroxide) in a third container, wherein the containers are packaged together in kit form for commercial, professional or individual consumer use. The present invention also contemplates formulated cream components, packages or kits for lye relaxer systems that instead include a lye relaxer, that is, an alkali metal hydroxide, preferably sodium hydroxide, in a container as a first formulated cream for hair straightening, with a second diluent cream of essentially equal viscosity, but without the alkali metal hydroxide, in a second container. The kits for lye relaxer systems might not include and do not require a separate aqueous activator solution. An embodiment of the present invention is a hair straightening kit that is configured for single, individual use. Another embodiment of the present invention is a hair straightening kit that is configured for multiple uses.

A conditioner may be added to the creams as desired, and appropriate conditioners are known in the art and will readily be identifiable to those of skill in the art. The formulations of the creams may also be adjusted as desired to include one or more surfactants, adjuvants, perfumes, emollients, moisturizers, colorants, or viscosity-modifying thinners or thickening agents.

The present invention also relates to methods for use of the hair straightening agents and diluent creams. A preferred embodiment of a no-lye relaxer method for straightening hair includes: mixing an aqueous activator solution containing guanidine carbonate with a relaxing precursor formulated cream containing calcium hydroxide to form an active hair straightening agent mixture with guanidine hydroxide as the effective active agent. An initial alkaline pH level of the mixture is set by the concentration of the relaxing precursor in the mixture and/or by the concentration of activator in this mixture, with more activator strengthening the mixture. An additional diluent cream of identical or comparable ingredients and identical or comparable viscosity to the relaxing precursor formulated cream, yet does not include calcium hydroxide or other relaxing precursor, is added to the mixture to dilute the strength of the activated relaxing agent prior to or at the time of application to a subject's hair. The no-lye relaxer method also provides for the use of other strong nitrogenous bases in the activator solution and the use of other alkali earth metal hydroxides in the relaxing precursor formulated cream. After a period of time determined by the manufacturer, as sufficient to have effected a desired degree of relaxation of the hair, the relaxer is rinsed from the hair, the remaining activated hair relaxer is neutralized with an alkali-neutralizing agent.

A preferred embodiment of a lye relaxer method for straightening hair includes: mixing a relaxer cream containing sodium hydroxide with a diluent cream of identical or comparable ingredients and identical or comparable viscosity to the relaxer cream, but which does not include sodium hydroxide or other lye or no-lye relaxing component, prior to application of the mixture to a subject's hair, so as to dilute the strength of the active lye relaxer. An initial alkaline pH level of the mixture is set by the concentration of the active relaxer in the mixture. After a period of time determined by the manufacturer, as sufficient to have effected a desired degree of relaxation of the hair, the relaxer is rinsed from the hair, the remaining activated hair relaxer is neutralized with an alkali-neutralizing agent. The lye relaxer method also provides for the use of other alkali metal hydroxides in the relaxer cream.

The time of treatment for straightening the subject's hair will typically be within a range of 5 minutes to 45 minutes. In most instances, the treatment time with a no-lye relaxer method will be at least 10 minutes, and while there is no fixed upper limit as to the time of treatment that the straightening agent mixture may remain on the subject's hair, usually the time of treatment will not extend beyond 30 minutes. With a lye relaxer method, the treatment times may be reduced, such that treatment times may last only a few minutes and will typically be between 10 and 18 minutes, and will usually not extend over 20 minutes. After the time for treatment has elapsed, the relaxing agent mixture should be removed completely from the hair to avoid potential damage or adverse effects to the subject's hair. The relaxing agent mixture may be removed by a thorough rinsing of the subject's hair with water. The rinse will be typically followed by a neutralizing step, which may be accomplished by the application of an alkali-neutralizing agent.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A system for straightening hair, comprising:
a first formulated relaxer cream containing an alkali metal hydroxide; and
a second formulated diluent cream of essentially equal viscosity to the relaxer cream, wherein the diluent cream does not include alkali metal hydroxide,
wherein the relaxer and diluent creams are suitable for being mixed to dilute concentration of the alkali metal hydroxide in the mixture of the creams and, further wherein the mixed relaxer and diluent creams are suitable to straighten hair upon application to hair.

2. The system of claim 1, wherein the alkali metal hydroxide is sodium hydroxide.

3. The system of claim 1, wherein the alkali metal hydroxide is lithium hydroxide.

4. The system of claim 1, wherein the system further comprises an alkali-neutralizing agent that is suitable for neutralizing the alkali metal hydroxide in the mixture upon application to hair, after the relaxer is rinsed from the hair.

5. A system for straightening hair, comprising:
an aqueous activator solution containing a strong nitrogenous base;
a first formulated precursor cream containing an alkali earth metal hydroxide; wherein neither the activator solution nor the precursor cream alone is operable to straighten hair, but when mixed produces an alkali of a strength sufficient to straighten hair; and
a second formulated diluent cream of essentially equal viscosity to the precursor cream, wherein the diluent cream does not include an alkali earth metal hydroxide,
wherein the activator solution, precursor cream and diluent cream are suitable for being mixed to produce an active hair straightening agent mixture that has an alkali concentration in the mixture that is less than the concentration of the alkali in a mixture without the diluent cream, and upon application the mixture straightens hair.

6. The system of claim 5, wherein the strong nitrogenous base is guanidine carbonate.

7. The system of claim 6, wherein the guanidine carbonate is of a concentration of 20-30% by weight in the activator solution.

8. The system of claim 7, wherein the guanidine carbonate is of a concentration of 25-29% by weight in the activator solution.

9. The system of claim 8, wherein the guanidine carbonate is of a concentration of about 27% by weight in the activator solution.

10. The system of claim 5, wherein the alkali earth metal hydroxide is calcium hydroxide.

11. The system of claim 10, wherein the calcium hydroxide is of a concentration of 4-10% by weight in the first formulated relaxer cream.

12. The system of claim 11, wherein the calcium hydroxide is of a concentration of about 6% by weight in the first formulated relaxer cream.

13. The system of claim 5, wherein the system further comprises an alkali-neutralizing agent that is suitable for neutralizing the active hair relaxing agent mixture upon application to hair, after the relaxer is rinsed from the hair.

14. A kit for preparing a composition for straightening hair, the kit comprising:
a first formulated relaxer or relaxing precursor cream comprising an alkali metal hydroxide or alkali earth metal hydroxide in a first container; and
a second formulated diluent cream of essentially equal viscosity to the relaxer or precursor cream without an alkali metal hydroxide or alkali earth metal hydroxide in a second container,
wherein the containers are packaged together in kit form for use.

15. The kit of claim 14, wherein the alkali metal hydroxide is sodium hydroxide.

16. The kit of claim 14, wherein the alkali earth metal hydroxide is calcium hydroxide.

17. The kit of claim 14, wherein the kit further comprises an aqueous activator solution comprising a strong nitrogenous base in a third container.

18. The kit of claim 14, wherein the kit is suitable for single use.

19. The kit of claim 14, wherein the kit is suitable for multiple use.

20. A method for straightening hair, comprising:
mixing an aqueous activator solution comprising guanidine carbonate with a formulated precursor cream comprising an alkali earth metal hydroxide to produce an active hair straightening agent admixture comprising guanidine hydroxide;
adding a diluent cream of essentially equal viscosity to the precursor cream, the cream not including an alkali earth metal hydroxide, to the admixture comprising guanidine hydroxide to dilute the concentration of the active straightening agent in the admixture prior to or during the time of application to a subject's hair; and
further wherein the admixture is applied to hair for a sufficient time period as to result in straightening of the hair.

21. The method of claim 20, further comprising after the sufficient period of time, neutralizing the active straightening agent on the hair.
